(19) United States
(12) Patent Application Publication
(10) Pub. No.: US 2006/0078530 A1
(43) Pub. Date: Apr. 13, 2006

(54) Using high-density grapes to rejuvenate human skin

(76) Inventor: Kay Miyakawa Liu, San Marino, CA (US)

Correspondence Address: Craig Miller, 642 Via Umbroso, San Clemente, CA, 92672, US

(21) Appl. No.: 10/961,305

(22) Filed: Oct. 12, 2004

(51) Int. Cl. A61K 36/87; A61K 36/48; A61K 31/12

(52) U.S. Cl. 424/74; 424/757; 424/766; 514/682

(57) ABSTRACT

The present invention provides a method for rejuvenating the skin by applying a chemical system that stimulates the skin to repair and build itself naturally at the same time the chemical system provides protection for the skin. More specifically, the stimulation of the skin is performed through the use of a Grape Botanical combined with a cosmetic system to, further stimulate and protect the skin.

### FIELD OF THE INVENTION

The present invention relates to a skin care product for preventing and rejuvenating the skin by using highly conductive oxidants. In the method a chemical system is delivered to the layers of the skin by use of an ultrasonic device or any other application method known in the art. Upon delivery of the chemical system past the dermal layers the system works with the metabolism to regenerate the skin.

### BACKGROUND OF THE INVENTION

For years various methods of rejuvenating the skin have been practiced. One method that is well-known in the art as microdermabrasion. Microdermabrasion uses highly controlled vacuum and pressure to move microscopically abrasive crystals over the surface of the skin. By repeating this process over the course of several weeks or months, the younger, softer skin moves to the surface, revealing significant textural and color changes. Another well-known practice for rejuvenating the skin involves invoice surgery to tighten the skin, which is also well-known in the art as facelift or rhytidectomy. Rhytidectomy can improve the most visible signs of aging by removing excess fat, tightening underlying muscles, and redraping the skin of your face and neck. When a facelift is performed by a qualified plastic surgeon, complications are infrequent and usually minor. Still, individuals vary greatly in their anatomy, their physical reactions, and their healing abilities, and the outcome is never completely predictable. Complications can occur due to the invasive process of surgery. Most common occurrences of complications include hematoma, which is a collection of blood under the skin that must be removed by the surgeon, injury to the nerves that control facial muscles, infection, and reactions to the anesthesia and poor healing of the skin.

While these efforts might work in the short term, they don't solve the underlying problem behind healthy skin—reinforcing the skin and preventing damage from the outside (stress, sun exposure, pollution, etc). The current invention overcomes the difficulties of current methods and procedures by protecting the skin from destroying factors, which are known in the art as free radicals, while stimulating the skin to repair and build itself naturally.

### SUMMARY OF THE INVENTION

The present invention provides a method for rejuvenating the skin by applying a chemical system that stimulates the skin to repair and build itself naturally at the same time the chemical system provides protection for the skin. More specifically, the stimulation of the skin is performed through the use of a Grape Botanical combined with a cosmetic system to, further stimulate and protect the skin.

Additionally, an advantage of the present invention is to provide a method of utilizing a Grape Botanical to scavenge free radical oxidants that are formed on the outer surface of the skin due to excessive sun exposure, age and negative environmental conditions. Antioxidants are molecules that interact with the free radicals thereby neutralizing them, which results in protecting normal tissue and DNA from potential damage. Once the antioxidants have neutralized the free radicals, they are either recycled or remain in a stable, altered state. Normal cellular respiration is not the only source of free radicals. Our environment exposes the body to a number of additional free radical sources, i.e. ionizing radiation (industry, the sun, cosmic rays), ozone and nitrous oxide primarily from auto exhaust emissions, heavy metals (lead, mercury, and cadmium), cigarette smoke both primary and secondary, and alcohol. When these free radicals enter the body, they can react with healthy tissues the same as the free radicals that are the result of cellular respiration, again with the potential to cause damage. It is believed that free radicals play a role in many different disease processes by causing oxidative stress. A recent Harvard University study profiled in Nature Magazine (September 2003), revealed research results from studies on Grape Botanicals. More specifically, it was found that special chemical compounds known as polyphenols effectively scavenge free radicals found in humans. The current invention uses extracts from Grape Botanicals, which we have found to contain more effective polyphenol known as resveratrol. Furthermore, resveratrol is highly effective at bonding with highly oxidized free radicals where the most common anti-oxidants such as Vitamin E or C fail. In addition, resveratrol works to stimulate human genes known as sirtuins, which help regulate the body's aging. In studies with yeast cells resveratrol stimulated the sirtuin gene activity nearly ten times over that of other similar ingredients, allowing the yeast cells to have a longer life span during testing. All of these effects mean that the resveratrol found in Grape Botanicals are powerful boosts to our body's natural anti-aging mechanisms.

### BRIEF DESCRIPTION OF THE DRAWINGS

FIG. 1 illustrates the layers of the skin

FIG. 2 illustrates the various forms of botanical grapes

FIG. 3 illustrates a graph of loss of resveratrol as heat increases

### DETAILED DESCRIPTION OF THE INVENTION

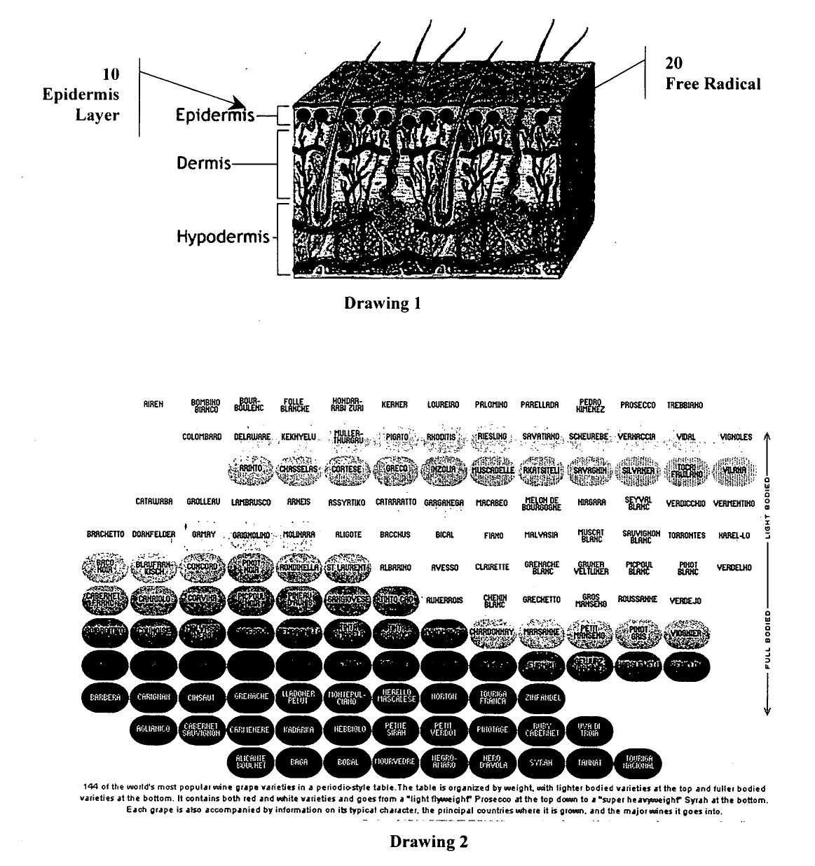

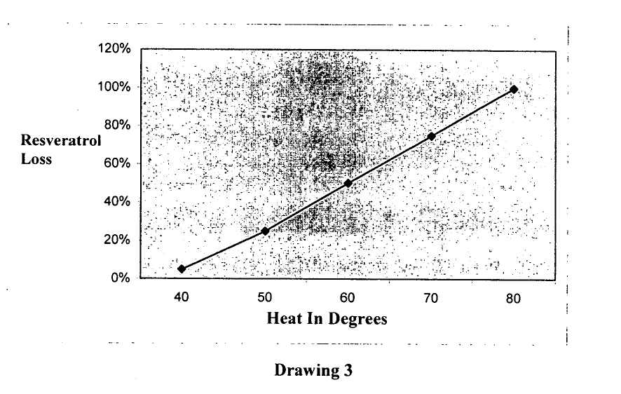

Due to the nature of Grape Botanicals their resveratrols are extremely heat-sensitive, and are almost completely destroyed in cooking. Therefore only relatively small amounts of resveratrols are available in our diet. Further compounding the problem is the limited supply of blood due to the lack of blood vessels in the epidermis layer 10 of the skin, as shown if FIG. 1. Thus, supplying a fast-acting and effective way of helping skin inhibit free radicals 20 is to provide resveratrols by applying them topically.

In the present invention the resveratrols are extracted from the Grape Botanicals through the well-known process of producing Red Wine. Grapes Botanicals are harvested and selected for manufacturing and distillation into wine. The grapes are then squeezed to extract all nutrients and juices. The juices and skins, known in the art as Must, are then stored for fermentation, which may take several weeks. The Must is sanitized in an extraction process that dilutes the water and alcohol from the juices. The remaining portion from the sanitation process, which is mostly sediment, is then mixed with Isoprene Glycol and non-ionic water is re-inserted. Water and any remaining alcohol are again diluted from the solution, and the remaining portion is allowed to sit until dry. Once dry the mixture is granulated several times into a fine powder called resveratrol amalgam. The resveratrol amalgam is further combined with various vitamins and minerals known in the art of cosmetology to protect skin.

In the preferred embodiment of the present invention extract from a Grape Botanical that has been processed as described herein to produce resveratrol amalgam is combined with a cosmetic system and applied to the skin. The method and process for combining the resveratrol amalgam with additional additives and chemicals is the need to keep the temperature during the mixing process below 40 degrees Celsius. It is common in the art to use heat to combine chemicals and agents

Due to resveratrol amalgam's sensitivity to heat which breaks down the resveratrol agent. Using heat to combine two chemical agents is well-practiced in the art of cosmetology. It is well-known in the art to use heat such that the current chemical bonds break down and form new chemical bonds as the two chemical agents are physically combined. In this embodiment of the present invention the cosmetic system is smartly configured based on a desired outcome. For example, if a topical lotion that contains sunscreen is desired the resveratrol amalgam is combined with an ultraviolet inhibitor. Ultraviolet inhibitors are well-known in the art. U.S. Pat. No. 6,280,712 describes the process for enhancing the effectiveness of a sunscreen lotion containing at least one ultraviolet filter involving solubilizing/dispersing the ultraviolet filter in a dialkyl/alkenyl carbonate. To minimize loss of resveratrol in the resveratrol amalgam heat is controlled to stay below 40 degrees Celsius. As show in FIG. 3 the addition of heat causes a significant loss in resveratrol.

The benefits achieved from combing the resveratrol amalgam with various ingredients known in the art to rejuvenate and protect the skin are significant and include: inhibit fatty acid oxidation and help to keep cell membranes in good condition; restore venous tone and help palliate “heaviness of the legs”; stimulate collagen and elastin synthesis in the skin thereby keeping it firm and elastic; inhibit the activity of various enzymes involved in inflammation—thereby soothing the skin and combating the ageing process; reduce melanin synthesis and inhibit tyrosinase activity, thereby lightening the skin, restoring the brightness of the complexion and preventing the appearance of pigmented spots; inhibit glycosylation processes, thereby cutting down the cross linking of collagen fibers which is responsible for so many of the phenomena associated with the ageing of the skin.

What is claimed is:

1. A rejuvenating skin chemical system comprising:
an resveratrol amalgam which is made from grape botanicals; and
a cosmetic system which contains vitamin C;
wherein chemical system is applied topically to the skin.

2. The revitalizing human skin system of claim 1, wherein the grape botanicals consist of gapes used to produce red wine.

3. The revitalizing human skin system of claim 1, wherein the cosmetic system contains vitamin K.

4. The revitalizing human skin system of claim 1, wherein the cosmetic system contains soy.

5. A rejuvenating skin chemical system comprising:
an resveratrol amalgam which is made from grape botanicals; and
a cosmetic system which contains a cosmetic system;
wherein chemical system and the cosmetic system are combined using heat with a temperature less than 40 degrees Celsius.

6. The revitalizing human skin system of claim 5, wherein the grape botanicals consist of gapes used to produce red wine.

7. The revitalizing human skin system of claim 5, wherein the cosmetic system contains vitamin K. The revitalizing human skin system of claim 5, wherein the cosmetic system contains soy.
